(19) United States
(12) Patent Application Publication
(10) Pub. No.: US 2024/0311565 A1
(43) Pub. Date: Sep. 19, 2024

(54) Methods and Systems for Identifying and Managing Biological Samples from Non-Human Subjects

(71) Applicant: IDEXX Laboratories, Inc., Westbrook, ME (US)

(72) Inventors: Jennifer Braff, Westbrook, ME (US); Kevin Knight, Westbrook, ME (US); Katie Goznikar, Westbrook, ME (US); Sarah Peterson, Westbrook, ME (US); Kristen Davenport, Westbrook, ME (US); Zenhwa Ouyang, Westbrook, ME (US)

(21) Appl. No.: 18/603,653

(22) Filed: Mar. 13, 2024

Related U.S. Application Data
(60) Provisional application No. 63/490,350, filed on Mar. 15, 2023.

(51) Int. Cl. G06F 40/279; G16H 10/60; G16H 70/20; G16H 70/40

(52) U.S. Cl. CPC G06F 40/279; G16H 10/60; G16H 70/20; G16H 70/40

(57) ABSTRACT

An example computer-implemented method for identifying biological samples from non-human subjects for testing includes receiving medical information of a non-human subject, receiving test results including a detected characteristic of a component indicative of a condition for a biological sample from the non-human subject, extracting one or more keywords from the medical information by executing a machine-learning natural language processing logic, and determining whether the detected characteristic is outside of a configurable threshold. In response to determining that the detected level is outside of the configurable threshold and based on the keywords being present in the medical information, the method includes generating instructions to retain the biological sample for further testing.

### CROSS REFERENCE TO RELATED APPLICATION

The present disclosure claims priority to U.S. provisional application No. 63/490,350, filed on Mar. 15, 2023, the entire contents of which are herein incorporated by reference.

### FIELD

The present disclosure relates generally to methods and systems for identifying and managing biological samples of interest.

### BACKGROUND

A variety of medical data is generated for a non-human subject during veterinary clinic visits. During the clinic visit, a veterinarian or veterinarian technician can take a sample from the non-human subject, such as a stool sample, blood sample, urine sample, fine needle aspirate sample or the like. The sample is tested and the test results can indicate if the non-human subject is suffering from a condition.

### SUMMARY

In one example, the sample may be tested for markers that are indicative of a condition. In some instances, characteristics of cells of the sample and/or components within the sample are identified via testing to assist in determining whether the non-human subject is suffering from a condition. However, some markers, characteristics, and/or components are not fully understood. For example, in some instances there is not an understood correlation between a condition and the presence of a specific marker.

In some instances, during the clinic visit the veterinarian or veterinarian technician performs a physical or visual assessment of the non-human subject. Sometimes an owner of the non-human subject provides information to the veterinarian or veterinarian technician based on what the owner has observed, such as changes in the non-human subject's behavior, eating habits, activity level, and the like. The veterinarian then writes down the medical observations of the non-human subject's condition (as observed directly or as relayed to the veterinarian through the owner) in the non-human subject's medical chart and may diagnose the non-human subject with a known condition based on the medical observations. However, in many instances, information in the medical chart is not necessarily associated with any markers or samples taken from the non-human subject. Accordingly, although known conditions might be identified through medical observations, lack of correlation between the medical observations and samples from the non-human subject can frustrate the identification of markers, characteristics, or components in the samples that might further assist in condition diagnosis.

Within examples, methods and systems are described to correlate information from clinic visits and information from test results of a sample taken from a non-human subject. The systems and methods include using a machine-learning natural language processing logic to determine if the biological sample should be retained. In some examples, a second machine-learning logic that is trained using labeled sample training data is used to determine a similarity between the biological sample and other biological samples within a catalog of biological samples.

In an example, a computer-implemented method for identifying biological samples from non-human subjects for retention is described. The method comprises receiving medical information of a non-human subject, the medical information comprising species, breed, and one or more clinical signs; receiving test results for a biological sample from the non-human subject, the test results comprising a detected characteristic indicative of a condition; extracting, by a processor executing a machine-learning natural language processing logic, one or more keywords from the medical information, wherein the machine-learning natural language processing logic is trained using labeled medical records training data; and determining whether the detected characteristic is outside of a configurable threshold. In response to determining that the detected characteristic is outside of the configurable threshold and based on the extracted one or more keywords matching one or more configurable keywords, the method also comprises generating instructions to retain the biological sample for further testing.

In another example, a computer-implemented method for identifying non-human subject candidates is described. The method comprises extracting, by a processor executing a machine-learning natural language processing logic, one or more keywords from medical information associated with the non-human subject, wherein the machine-learning natural language processing logic is trained using labeled medical records training data, and wherein the medical information comprises species, breed, and one or more clinical signs; receiving test results associated with the non-human subject, the test results comprising a detected characteristic indicative of a condition; determining whether the extracted one or more keywords comprise a configurable keyword; and determining whether the detected characteristic is outside of a configurable threshold. In response to determining the extracted one or more keywords comprises the configurable keyword and the detected characteristic is outside of the configurable threshold the method also comprises, assigning a non-human subject identifier to the non-human subject; and generating instructions to direct future biological samples from the non-human subject for further analysis based on the non-human subject identifier.

In another example, a server is described comprising one or more processors, and a non-transitory computer readable medium having stored therein instructions that when executed by the one or more processors, causes the server to perform functions. The functions comprise receiving medical information of a non-human subject, the medical information comprising species, breed, and one or more clinical signs; receiving test results for a biological sample from the non-human subject, the test results comprising a detected characteristic indicative of a condition; extracting, by the one or more processors executing a machine-learning natural language processing logic, one or more keywords from the medical information, wherein the machine-learning natural language processing logic is trained using labeled medical records training data; and determining whether the detected characteristic is outside of a configurable threshold. In response to determining that the detected characteristic is outside of the configurable threshold and based on the extracted one or more keywords comprising one or more configurable keywords the functions also comprise, generating instructions to retain the biological sample for further testing.

The features, functions, and advantages that have been discussed can be achieved independently in various examples or may be combined in yet other examples. Further details of the examples can be seen with reference to the following description and drawings.

### BRIEF DESCRIPTION OF THE FIGURES

Examples and descriptions of the present disclosure will be readily understood by reference to the following detailed description of illustrative examples when read in conjunction with the accompanying drawings, wherein:

FIG. 1 illustrates an example of a system, according to an example implementation.

FIG. 2 illustrates an example of a computer system of the system in FIG. 1, according to an example implementation.

FIG. 3 illustrates an example of a generalized workflow utilizing a medical information database of the computer system of FIG. 2, according to an example implementation.

FIG. 4 illustrates an example workflow of functions described in FIG. 3, according to an example implementation.

FIG. 5 illustrates an example of sample medical information of the generalized workflow in FIG. 3, according to an example implementation.

FIG. 6 shows a flowchart of an example of a method for identifying biological samples from non-human subjects, according to an example implementation.

FIG. 7 shows a flowchart of an example of a method for identifying non-human subject candidates, according to an example implementation.

### DETAILED DESCRIPTION

Disclosed examples will now be described more fully hereinafter with reference to the accompanying drawings. Several different examples may be described and should not be construed as limited to the examples set forth herein. Rather, these examples are described so that this disclosure will be thorough and complete and will fully convey the scope of the disclosure to those skilled in the art.

Systems and methods described herein include a computer-implemented method for identifying biological samples. In embodiments, the test results include a detected characteristic that is indicative of a condition. The medical information, in embodiments, is processed by executing a machine-learning natural language processing logic that is trained using labeled medical records training data to extract keywords from the medical information. The test results are processed to determine whether the detected characteristic outside of a configurable threshold. Based on the keywords that are extracted from the medical information, and in response to determining that the detected characteristic is outside of the configurable threshold, instructions are generated to retain the biological sample for further testing.

In some embodiments, systems and methods described include a computer-implemented method for identifying non-human subject candidates. In embodiments, medical information is processed by a machine learning natural language processing logic that is trained using labeled medical records training data, to extract keywords from the medical information. The extracted keywords are processed to determine whether one or more keywords include a configurable keyword. Test results associated with the non-human subject include a detected characteristic indicative of a condition are received and processed to determine whether the detected characteristic is outside of a configurable threshold. A non-human subject identifier is assigned to the non-human animal non-human subject in response to determining that the extracted one or more keywords comprise the configurable keyword and the detected characteristic is outside of the configurable threshold.

Implementations of this disclosure provide technological improvements that are particular to computer technology, for example, those concerning operation and utilization of machine-learning algorithms. Computer-specific technological problems, such as training algorithms to determine what data to extract, can be wholly or partially solved by implementations of this disclosure. For example, implementation of this disclosure allows for unstructured medical information to be structured and searched and keywords extracted based on a natural language processing logic.

The systems and methods of the present disclosure further address problems particular to computer devices and operation of diagnostic instruments, for example, those concerning analysis of medical information and biological sample test results. Utilizing machine-learning algorithms, trained on labeled medical records data, enables a more immediate and normalized analysis of the information. Thus, analysis of the medical information can occur in a manner that is efficient and takes into account all a non-human subjects' medical history and results utilizing medical information and test results are provided in a manner allowing for immediate determination whether the biological sample should be retained for further testing. Implementations of this disclosure can thus introduce new and efficient improvements in the ways retainment of biological samples are determined by the central computing device for use of the diagnostic instruments in an efficient manner.

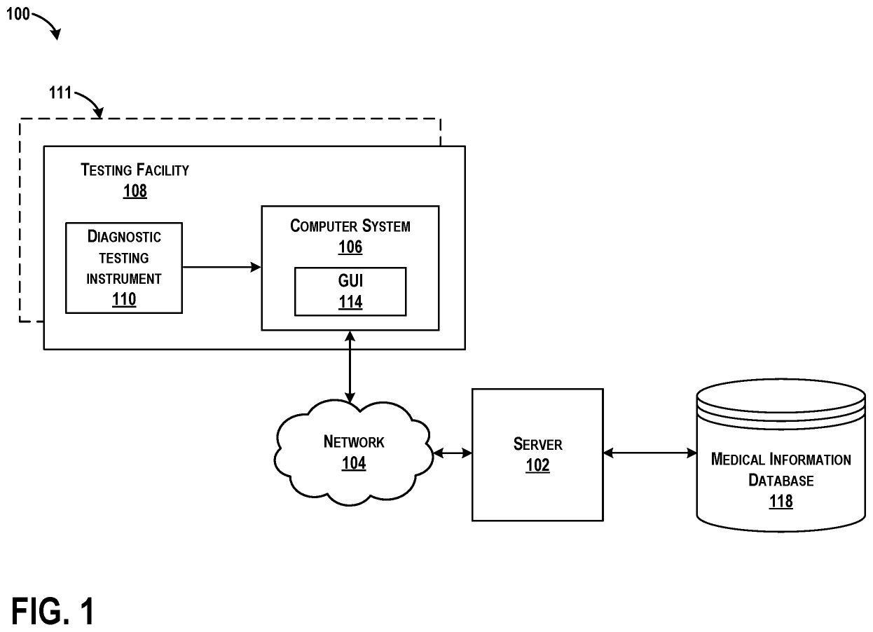

Referring now to the figures, FIG. 1 illustrates an example of a system 100, according to an example implementation. The system 100 includes a server 102 accessible through a network 104 by computer systems. One computer system includes a computer system 106 residing at a testing facility 108. In embodiments, the testing facility 108 includes a diagnostic testing instrument 110 configured to perform diagnostic testing of veterinary non-human subjects, for example. The diagnostic testing instrument 110 outputs test results to the computer system 106 for analysis. While one testing facility 108 is depicted in FIG. 1, it should be understood that this is merely an example, and systems according to the present disclosure can include any suitable number of testing facilities and associated computer systems, such as a second testing facility 111 that includes similar or the same components as the testing facility 108. As referred to herein, the term “testing facility” includes any entity at which samples from non-human animals are tested, such as reference laboratories or point-of-care facilities, and can include brick and mortar locations, pop-up clinics, mobile clinics, and the like.

While the example depicted in FIG. 1 includes one diagnostic testing instrument 110, it should be understood that this is merely an example, and embodiments according to the present disclosure can include any suitable number of diagnostic testing instruments associated with the testing facility 108. Examples of the diagnostic testing instrument 110 include any one or combination of veterinary analyzers operable to conduct a diagnostic test of a sample of a non-human subject (e.g., operable to determine hemoglobin amounts in a blood sample, operable to analyze a urine sample, and/or the like). Such veterinary analyzers include, for example and without limitation, a chemistry analyzer, a hematology analyzer, a urine analyzer, an immunoassay reader, a sediment analyzer, a blood analyzer, a digital radiology machine, a microscopy analyzer, and/or the like. In one example, the computing system 106 is in communication with a veterinary analyzer of the diagnostic testing instrument 110 and is operable to receive diagnostic information from veterinary analyzer. The diagnostic testing instrument 110 outputs signals, such as signals indicative of diagnostic test results or other information, to the computing system 106.

In embodiments, the computer system 106 receives an input from the diagnostic testing instrument 110, and the input includes test result data for a biological sample associated with a non-human subject. The data is associated with test results output from the diagnostic testing instrument 110 and includes a characteristic indicative of a condition. In some embodiments, the characteristic indicative of the condition is a detected level of a component, such as a marker, an antigen, an antibody or the like. In some embodiments, the characteristic indicative of the condition is a presence and/or a concentration of various components in a sample, such as ova and parasites or the like. In some embodiments, the characteristic indicative of the condition is one or more properties of cells within the sample, such as cell morphology or the like.

What constitutes a detected characteristic indicative of the condition can vary depending on a number of factors such as the characteristic being tested, the testing equipment being used, and type of test being performed. If a component is detected and/or capable of being measured by the test, then the component can be considered a detected level. Examples of components being detected include, for example and without limitation, complete blood counts (CBCs), sample morphology analysis, urinalysis parameters (e.g., concentrations of analytes in the urine), comprehensive metabolic panels (e.g., glucose, sodium, potassium, creatinine, etc.), multiplexed infectious disease panels that detect infectious antigens and/or presence of antibodies to infectious agents, markers (e.g., fructosamine). Thus, any level or any amount of these components that is an amount possible of being detected is considered a detected level. Within examples, the detected level of the component indicative of the condition is used to filter out test results that are otherwise not indicative, in any way, of the condition under consideration. In one example, the detected level of the component indicative of the condition comprises a detected level of markers, including alanine transaminase (ALT).

The system 100, in embodiments, includes a medical information database 118. The medical information database 118 stores medical information of non-human subjects, such as may be gathered from one or more testing facilities such as testing facility 108. The medical information includes species, breed, one or more clinical signs, medications, and/or other similar non-human subject record information associated with the non-human animal subject. In some embodiments the medical information database 118 includes records associated with the non-human animal subject contained within a practice information management system (PIMS). The computer system 106 receives data from the medical information database 118, via the network 104, and the data includes medical record information associated with a veterinary non-human subject and a veterinary non-human subject identifier unique to the veterinary non-human subject. While in the embodiment depicted in FIG. 1, the computer system 106 is communicatively coupled to the medical information database 118 via the network 104 and the server 102, it should be understood that medical information database 118 and the computer system 106 can be directly communicatively coupled to one another via the network 104 (i.e., without the intervening server 102).

In some examples, the computer system 106 includes a graphical user interface (GUI) 114 for display. In some embodiments, the GUI 114 is operable to receive inputs at the graphical user interface 114. While in the embodiment depicted in FIG. 1, the computer system 106 includes the GUI 114, it should be understood that this is merely an example, and the computer system 106 can include any suitable user interface for receiving data input, for example and without limitation, an alphanumeric keyboard or the like. In one example, clinical information, such as information regarding a dose of medication provided to the subject and/or information relating to clinical and/or historical observations of the subject, is input into a clinical decision support interface on GUI 114 of computer system 106. The clinical information is then stored, after processing, within the medical information database 118. Thus, the medical information includes the clinical information input into the clinical decision support interface on GUI 114.

In the system 100, the network 104 (e.g., Internet) provides access to the server 102 for all network-connected components. In some examples, more components of the system 100 may be in communication with the network 104 to access the server 102. Communication with the server 102 and/or with the network 104 may be wired or wireless communication (e.g., some components may be in wired Ethernet communication and others may use Wi-Fi communication).

While in some examples, the system 100 enables a method for identifying biological samples from non-human subjects for testing. In some examples the system 100 enables a method for identifying non-human subject candidates. In embodiments, the computer system 106 receives inputs from the diagnostic testing instrument 110. In some examples, biological sample test result data associated with a non-human subject is input into the graphical user interface 114 of computer system 106. Within examples, the biological sample test result data includes any of outputs from the diagnostic testing instrument 110, other test results as well of medical tests performed on the non-human subject and/or the like. The computer system 106, via communication with the diagnostic testing instrument 110, receives test result data associated with the non-human subject, and the test result data includes a detected characteristic that is indicative of a condition.

The computer system 106, receives medical information data associated with the non-human subject, via access to the medical information database 118 through the server 102 and/or via data input via GUI 114. Within examples, the medical information data includes information of the non-human subject (e.g., species, breed, and one or more clinical signs), pathology reports, medications the non-human subject is currently taking, and any other medical information associated with the non-human subject.

The test result data and medical information data is received by the computer system 106 for processing. Example processing includes extracting a keyword from the medical information, determining whether the detected characteristics from the test results is outside of a configurable threshold, and generating instructions to retain the biological sample for further testing based on the keywords and the detected characteristic being outside of the configurable threshold. In one example, the keywords to be extracted and the configurable threshold are received from a remote computing device. The determination whether to retain a biological sample for further testing includes an analysis of the medical information, based on the extracted keyword, to provide indications that the sample is of interest, such as an indication that a disease or pathogen is likely present.

In some instances, the configurable threshold is an established reference range in which the detected characteristic exists for healthy subjects. For example, in instances in which a detected level of the component is outside of (e.g., above or below) the configurable threshold, thus outside of the established reference range for healthy subjects, the detected level can indicate the subject has the condition under consideration. As an example, instructions are generated to retain the biological sample if the detected level for circulating symmetric dimethylarginine concentration is above the established reference range. Similarly, in some embodiments, instructions are generated to retain the biological sample if the detected level for circulating hemoglobin concentration is below the established reference range. In some instances, the configurable threshold is a medical decision point (e.g., a point and/or a value or a series of values in which a medical professional would take some action), outside of which the instructions are generated to retain the biological sample.

Instructions to retain a biological sample are determined by executing a machine-learning natural language processing logic, based on one or more keywords extracted from the medical information, where the machine-learning natural language processing logic is trained using labeled medical records training data (described more fully below). In examples in which the system 100 enables the method for identifying non-human subject candidates, processing includes assigning a non-human subject identifier to the non-human subject, and generating instructions to direct future biological samples from the non-human subject for further analysis based on the non-human subject identifier.

In one example, the test results are associated with a test performed at a first testing location, such as testing facility 108. In response to determining that the detected characteristic is outside of the configurable threshold and based on the extraction of one or more keywords in the medical information, the computer system 106 generates instructions to send a portion of the biological sample from the first testing location to a separate second testing location, such as testing facility 111. Testing facilities can vary in capabilities and available diagnostic testing instruments. By directing the portion of the biological sample to the second testing location, such as a research facility, a more complete and thorough analysis of the biological sample is able to be performed.

The GUI 114 of the computer system 106 displays instructions to retain the biological sample for further testing, send a portion of the biological sample to a second testing facility, and/or to direct future biological samples for testing. In some examples, the computer system 106 generates an electronic consent form for a customer associated with the non-human subject.

In some embodiments, the biological sample associated with the test results sent from the diagnostic testing instrument 110 to the computer system 106 includes a sample identifier. After the computer system 106 extracts keywords from the medical information and determines whether the detected characteristic is outside of the configurable threshold, the computer system 106 stores data with the test results. In particular, the computer system 106 stores data associating the sample identifier, such as the keywords extracted from the medical information. The data with the identifier is stored within a database, for example the medical information database 118. In embodiments, the medical information database 118 includes data from multiple samples, such that the medical information database 118 includes a catalog of data associated with biological samples. Thus, the data is added to the bank of data in a catalogued and standardized way. Future analysis may be able to add to, and/or pull from, the standardized and catalogued bank of data.

The system 100 provides advantages for the identification of biological samples that contain indications of a condition but lack a definite diagnostic code. In this way, the system 100 can form a more robust pool of biological samples for future analysis. For example, by identifying samples in which medical information may indicate a disease, a musculoskeletal condition, or pathogen is likely present but has not yet been confirmed with a diagnosis, the system 100 is able to expand beyond reliance on confirmed diagnostic cases and include other relevant information for correlation.

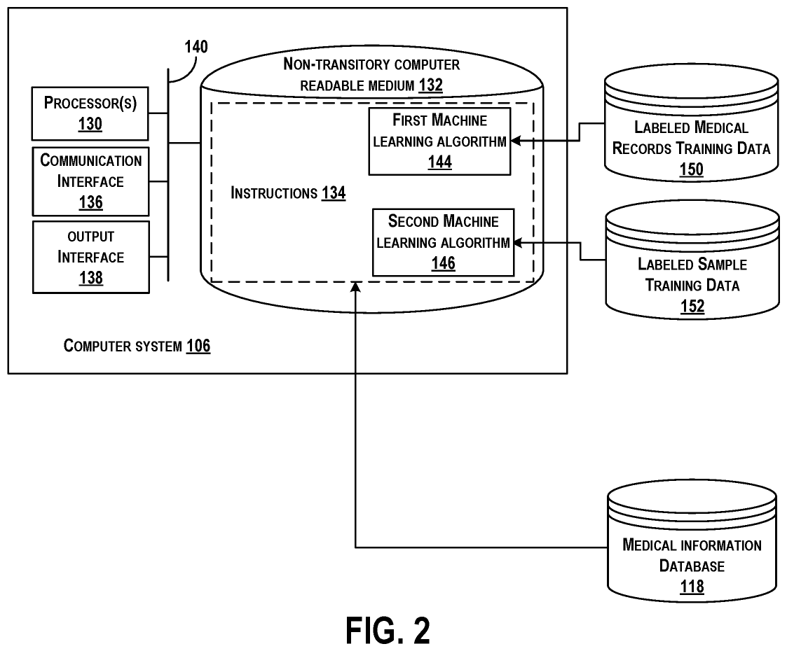

FIG. 2 illustrates an example of the computer system 106 in FIG. 1, according to an example implementation. Within examples herein, functions described for identifying non-human subject candidates are performed by the computer system 106, by the server 102, or by a combination of the computer system 106 and the server 102. Thus, although FIG. 2 illustrates the computer system 106, in some embodiments the components of the server 102 are similar or the same as the components of the computer system 106 and the illustration in FIG. 2 additionally represents components of the server 102, for example, depending on where a function is programmed to be performed in a specific example.

The computer system 106 includes one or more processor(s) 130, and a non-transitory computer readable medium 132 having stored therein instructions 134 that when executed by the one or more processor(s) 130, causes the computer system 106 to perform functions for operation, management, and control of diagnostic instruments, and for identifying non-human subject candidates, for example.

In embodiments, the computer system 106 includes a communication interface 136, an output interface 138, and components of the computer system 106 are connected to a communication bus 140. The computer system 106, in some embodiments, includes hardware to enable communication within the computer system 106 and between the computer system 106 and other devices (not shown). The hardware may include transmitters, receivers, and antennas, for example. The computer system 106 may further include a display.

In some embodiments, the communication interface 136 is a wireless interface and/or one or more wireline interfaces that allow for both short-range communication and long-range communication to one or more networks or to one or more remote devices. Such wireless interfaces may provide for communication under one or more wireless communication protocols, Bluetooth, WiFi (e.g., an institute of electrical and electronic engineers (IEEE) 802.11 protocol), Long-Term Evolution (LTE), cellular communications, near-field communication (NFC), and/or other wireless communication protocols. Such wireline interfaces may include an Ethernet interface, a Universal Serial Bus (USB) interface, or similar interface to communicate via a wire, a twisted pair of wires, a coaxial cable, an optical link, a fiber-optic link, or other physical connection to a wireline network. Thus, the communication interface 136 may be configured to receive input data from one or more devices, and may be configured to send output data to other devices.

The non-transitory computer readable medium 132 includes or takes the form of memory, such as one or more computer-readable storage media that can be read or accessed by the one or more processor(s) 130. The non-transitory computer readable medium 132 includes volatile and/or non-volatile storage components, such as optical, magnetic, organic or other memory or disc storage, which can be integrated in whole or in part with the one or more processor(s) 130. In some examples, the non-transitory computer readable medium 132 is implemented using a single physical device (e.g., one optical, magnetic, organic or other memory or disc storage unit), while in other examples, the non-transitory computer readable medium 132 is implemented using two or more physical devices. The non-transitory computer readable medium 132 thus is a computer readable storage, and the instructions 134 are stored thereon. The instructions 134 include computer executable code.

The one or more processor(s) 130 are general-purpose processors or special purpose processors (e.g., digital signal processors, application specific integrated circuits, etc.). The one or more processor(s) 130 receive inputs from the communication interface 136 (e.g., x-ray images), and process the inputs to generate outputs that are stored in the non-transitory computer readable medium 132. The one or more processor(s) 130 are configured to execute the instructions 134 (e.g., computer-readable program instructions) that are stored in the non-transitory computer readable medium 132 and are executable to provide the functionality of the computer system 106 described herein.

The output interface 138 outputs information for transmission, reporting, or storage, and thus, the output interface 138 may be similar to the communication interface 136 and can be a wireless interface (e.g., transmitter) or a wired interface as well.

In FIG. 2, the computer system 106 is shown to also be in communication with the medical information database 118. In this way, medical information including species, breed, and one or more clinical signs, is received at the computer system 106 from the medical information database 118.

In some instances, the medical information database 118 includes a database of a PIMS. Thus, the medical information of the non-human subject can be found by the processor 130 of computer system 106 searching within the database of the PIMS for information in a record associated with the non-human subject to extract one or more keywords. For example, the processor 130 can use identifying information about the non-human subject to search the PIMS database within the medical information database 118. For example, the processor 130 can search for keywords from the non-human subject's condition information indicative of a disease (e.g., “hypertension”, “elevated bp”, “elevated mmHg”) or indicative of a non-disease (e.g., “wellness”) to then use for identifying and locating samples to run a test on. The test results for the identified sample are then compared to data from diseased and non-diseased subjects to determine if a difference is detected. In some examples, the computer system 106 and the medical information database 118 can be directly communicatively coupled to one another via the network 104 (i.e., without the intervening server 102). In addition, while the medical information database 118 is depicted as a single database in the Figures, in other examples, it should be understood that the medical information database 118 includes or comprises multiple separate databases, cloud storage, or the like where radiology data is stored in a first database and medical information of patients is stored in a second database.

In another example, the medical information of the non-human subject can be found by the processor 130 of computer system 106 searching within the PIMS for prescription information of medication prescribed to the non-human subject to extract one or more keywords. For example, the processor 130 can search the PIMS database for prescription medication keywords. Without being bound by theory, prescriptions are used to treat certain conditions (e.g., NSAID, such as Carprofen, typically being used to treat arthritis). By mapping conditions to prescription information, researchers are provided with more data from which to study and monitor the efficacy of certain treatment options over time. Further, in some instances, prescription information can be indicative of a condition. Thus, prescription information can aid in the identification of subjects having certain conditions absent a definitive diagnosis being found in the subject's medical records.

The instructions 134 are executed by the processor 130 to determine whether the detected characteristic is outside of a configurable threshold. If the extracted one or more keywords include the configurable keyword and the detected level is outside of the configurable threshold, instructions are generated to retain the biological sample for further testing. In one example, instructions are generated to direct future biological samples from the subject for further analysis. The future biological samples can be directed to another facility, such as a central testing/research facility and/or central biological storage facility. By directing the future biological samples for further analysis, progression of the detected characteristic over time, and thus the potential appearance or progression of a condition, is better monitored. Additionally, more sample data is able to be generated and catalogued in a standardized way that allows for future mining of the data.

Additionally, in some examples if it is determined that extracted one or more keywords include the configurable keyword and the detected level is outside of the configurable threshold the computer system 106 identifies a blood sample from the same non-human subject from which the biological sample was collected. The blood sample data is stored within the medical information database 118 accessible by the computer system 106. Once the blood sample is identified, the computer system 106 generates instructions to retain the blood sample for further testing. In this instance, the physical blood sample is stored in the reference lab and indexed by a unique identifier to the subject. This can further aid in accelerating research and design by mapping the keywords and detected level to an already existing sample within the subject's medical records.

Within examples, the instructions 134 include specific software for performing the functions described above including a first machine-learning algorithm 144 (e.g., executable for determining one or more keywords from medical information), and a second machine-learning algorithm 146 (e.g., executable for determining a similarity between the biological sample and other biological samples in a catalog of biological samples).

The first machine-learning algorithm 144 is a machine-learning natural language processing logic that is trained using labeled medical records training data 150. The processor 130 executes the first machine-learning algorithm 144 to extract keywords from the medical information contained in the medical information database 118. The first machine-learning algorithm 144 is presented with the labeled medical records training data 150 that trains the first machine-learning algorithm 144 on keywords to extract from the medical information to determine whether keywords in newly received medical information include a configurable keyword. Thus, the first machine-learning algorithm 144 is executable to receive information from the medical information database 118 and determine whether one or more keywords present in the medical information include a configurable keyword. In some examples, the first machine-learning algorithm 144 can use the labeled medical records training data 150 to determine the configurable keyword(s). The first machine-learning algorithm 144 then receives information from the medical information database 118 and determines whether one or more keywords present in the medical information include the configurable keyword(s). Thus, the first machine-learning algorithm 144 can determine what configurable keyword(s) to search for in the received medical information.

In one example, the keywords extracted by the first machine-learning algorithm 144 include a diagnostic code. Test results from biological samples sometimes allow pathologists to assign a standard diagnosis. In labeling the subject with the diagnosis, pathologists can assign a diagnostic code to the test results. The first machine-learning algorithm 144 extracts the diagnostic code as a keyword. The diagnostic code, which is assigned to a diagnosis of the subject, is then associated with the medical information of the subject, such as the condition information. In some examples, the computer system 106 associates the diagnosis of the biological sample with the condition by referencing the diagnosis against a referential database. In some examples the computer system 106 performs association by referencing a lookup table of keyword conditions associated with the diagnosis. Other association techniques can also be used. These types of association allow a broader reference pool of medical data to be considered and thus provides a more complete understanding of the interplay between the medical information.

In one example, the processor 130 of the computer system 106 executes a second machine-learning logic, such as the second machine-learning algorithm 146, to determine a similarity between the biological sample and other biological samples in the catalog of biological samples. The second machine-learning algorithm 146 is trained using labeled sample training data 152. For the other biological samples having the similarity, the processor 130 stores within the database, such as the medical information database 118, data with identifiers of the other biological samples that identifies the other biological samples as being associated with the condition. Thus, the system is able to update and characterize other biological samples with data based on learned information from the current biological sample, providing a more robust association between medical information.

Referring to the first machine-learning algorithm 144 and the second machine-learning algorithm 146, many types of functionality and neural networks can be employed to perform functions of the machine-learning algorithms. In one example, the first machine-learning algorithm 144 and the second machine-learning algorithm 146 use statistical models to generate the outputs without using explicit instructions, but instead, by relying on patterns and inferences by processing associated training data.

The first machine-learning algorithm 144 and the second machine-learning algorithm 146 can thus operate according to machine-learning tasks as classified into several categories. In supervised learning, the first machine-learning algorithm 144 and the second machine-learning algorithm 146 build a mathematical model from a set of data that contains both the inputs and the desired outputs. The set of data is sample data known as the “training data,” in order to make predictions or decisions without being explicitly programmed to perform the task. For example, the first machine-learning algorithm 144 utilizes the medical records training data 150 and the second machine-learning algorithm 146 uses labeled sample training data 152.

In another category referred to as semi-supervised learning, the first machine-learning algorithm 144 and the second machine-learning algorithm 146 develop mathematical models from incomplete training data, where a portion of the sample input does not have labels. A classification algorithm can then be used when the outputs are restricted to a limited set of values.

In another category referred to as unsupervised learning, the first machine-learning algorithm 144 and the second machine-learning algorithm 146 build a mathematical model from a set of data that contains only inputs and no desired output labels. Unsupervised learning algorithms are used to find structure in related training data, such as grouping or clustering of data points. Unsupervised learning can discover patterns in data and can group the inputs into categories.

Alternative machine-learning algorithms may be used to extract keywords from the medical information and/or determine the similarity between the biological sample and other biological samples in the catalog of biological samples, such as deep learning though neural networks or generative models. Deep machine-learning may use neural networks to analyze medical information data through a collection of interconnected processing nodes. The connections between the nodes may be dynamically weighted. Neural networks learn relationships through repeated exposure to data and adjustment of internal weights. Neural networks may capture nonlinearity and interactions among independent variables without pre specification. Whereas traditional regression analysis requires that nonlinearities and interactions be detected and specified manually, neural networks perform the tasks automatically.

Still other machine-learning algorithms or functions can be implemented to generate the keywords. Support vector machine, Bayesian network, a probabilistic boosting tree, neural network, sparse auto-encoding classifier, or other known or later developed machine-learning algorithms may be used. Any semi-supervised, supervised, or unsupervised learning may be used. Hierarchal, cascade, or other approaches may be also used.

The first machine-learning algorithm 144 and the second machine-learning algorithm 146 may thus be considered an application of rules in combination with learning from prior data to identify appropriate outputs. Analyzing prior data allows the first machine-learning algorithm 144 and the second machine-learning algorithm 146 to learn patterns of keywords that are generally present in medical information associated with the biological sample that has a detected level indicative of a condition, for example.

Thus, the first machine-learning algorithm 144 and the second machine-learning algorithm 146 take the form of one or a combination of any of the herein described machine-learning functions, for example.

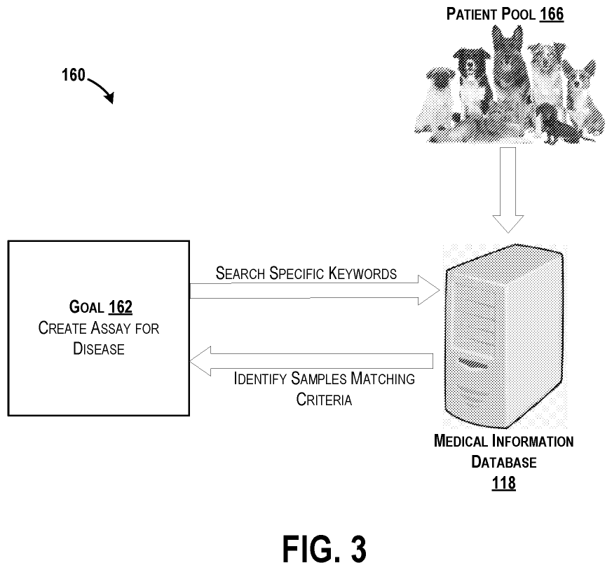

FIG. 3 illustrates an example of a generalized workflow 160 for identifying samples, according to an example implementation. During research and development, it is often advantageous to draw from existing data to reduce development time. When a new test is being developed for a condition, samples can be taken from non-human subjects having the condition. For example, samples from non-human subjects having a condition can be used to identify markers that are associated with the condition. In some examples, the marker can be a biomarker. Currently in the non-human health industry, many samples are not retained, making it difficult find samples from non-human subjects having a specific condition for research. The generalized workflow 160 provides one example in which data can be mined for sample data from non-human subjects exhibiting certain conditions of interest.

In the generalized workflow 160, a goal 162 is stated to create a test for a condition by searching the medical information database 118 of a non-human subject pool 166. The non-human subject pool 166 is made up of non-human animals; in this example dogs are depicted. When a non-human subject receives medical attention, such as from a veterinarian, medical information is gathered on the non-human subject. The medical information can be, test results, written records from a physical, etc. The medical information is then stored in an electronic medium, such as the medical information database 118 which is depicted as a server. In creating a test for a condition, keywords that are associated with the condition are used to search the medical information database 118 for matching samples from the non-human subject pool 166. Identified samples that match the keyword search criteria are then returned for mapping to the condition. A common characteristic between the identified samples can be associated with the condition and the test can be developed around the common characteristic. In some examples, a machine learning algorithm can be used to identify the common characteristic and/or a common pattern between the identified samples. For instance, a new marker for the condition can be found by using machine learning to determine common profiles and/or patterns between the identified samples.

In some examples, information that is useful in diagnosing a non-human subject is found in condition information sections of the subject's medical information. Condition information can include quantitative observations (e.g., blood pressure) and/or qualitative observations (e.g., abrasion) by the veterinarian or customer (e.g., a pet owner), or a combination of quantitative and qualitative observations (e.g., review of radiology data such as x-ray images and accompanying reports). Condition information is often taken during a physical exam of the subject and manually input by a veterinarian or veterinarian technician. For example, the condition information can be found in handwritten notes by a veterinarian that are transcribed onto an electronic storage medium within the subject's medical file. As an illustrative example, blood pressure is assessed during the physical exam of the subject rather than through analysis of a patient sample. The veterinarian assessing blood pressure includes the condition information, such as blood pressure value and/or observational conclusions such as “hypertension”, “elevated bp”, “elevated mm/Hg”, or “WNL (within normal limits)”, in the subject's medical records, which are stored within the medical information database 118. The condition information is often useful by correlating it to other medical information, such as test results from a biological sample to formulate a diagnosis.

In another example, it might be desirable to match a diagnostic code with sample test results from the same subject to correlate characteristics of the sample with a confirmed diagnosis. This process would provide the advantage of having the ability to pull data from a larger pool of samples of interest in the absence of biopsy data based on searching for other samples that have similar attributes to the sample of interest. In this instance, the computer system 106 of FIG. 2 uses the diagnostic code to match to samples from the same subject. After locating the sample test results for the subject, the computer system 106 analyzes the medical data to extract relevant keywords. The first machine-learning algorithm 144 uses the extracted keywords to evaluate similar medical data that may not have an assigned diagnostic code. In other words, the first machine-learning algorithm 144 uses the medical information from the subject that has the diagnostic code to determine configurable keywords to assist in identifying medical information from non-human subjects having a similar condition. The first machine-learning algorithm 144 can match a similar constellation of test results (e.g., detected characteristics), similar non-human subject demographic information, and/or other similar features, for example.

In some instances, medications used to treat one condition of a non-human subject affect the parameters of test result data that is used to analyze another condition. Some medications have cross-reactant potential which ultimately skews test result parameter interpretation. For example, medications commonly used to treat Cushing's disease are known to increase the concentration of the cross-reactant for the analyte of interest (e.g., cortisol). Knowing the diagnosis (e.g., Cushing's or no Cushing's) and treatment status (e.g., receiving treatment or not receiving treatment) allows analysis to account for test result data being skewed by medications.

In one example, the information can be obtained based on input into a clinical decision support (CDS) interface. Keeping with the Cushing's example, when veterinarians order a diagnostic test, such as a low-dose dexamethasone-suppression test (LDDST), the GUI 114 in FIG. 1 displays CDS input cards to the veterinarian with prompts regarding the test and about clinical signs. Input received in response to the CDS prompts provide information from which a likelihood of Cushing's disease diagnosis can be determined, and the prompts capture information from the veterinarian about the dose of medication provided to the non-human subject and information relating to clinical and/or historical observations in the non-human subject. Further, the CDS input can be used in labeling samples with the likely diagnosis and the treatment status. Thus, groups of non-human subjects can be formed from the likely diagnosis and treatment status (e.g., diseased-treated, diseased-untreated, non-diseased-untreated, etc.) and the concentration of the cross-reactant of concern is testable to ascertain risk level.

In another example, it is desirable to monitor liver disease progression over time, but to do so requires multiple samples from a non-human subject collected over time. While analysis of blood samples can identify the presence of liver disease, it cannot at this time identify the type of liver disease. Pathology reports can be searched based on keywords that would increase the likelihood of a diagnosis of various liver conditions (e.g., “hepatitis”, “hepatopathy”, “cirrhosis”, “fibrosis+liver”, etc.). Based on the keyword search, non-human subjects can be automatically identified and flagged for sample acquisition and monitoring with respect to liver disease, in addition to past samples of the non-human subject being located and tested. The gathered data can allow for a test to be created which detects specific liver diseases, and/or one used to monitor liver disease progression or treatment over time.

In summary, currently, samples are not catalogued in a standardized way making identification of samples of interest difficult. There is a need to locate samples from non-human subjects having certain conditions to identify characteristics that can enable early detection of the condition and/or assist in developing treatment regimes. The above examples described with respect to FIG. 3 are illustrative of the ability to mine current medical information data of interest to create characterizations for the samples and generate a standardized catalogue of samples.

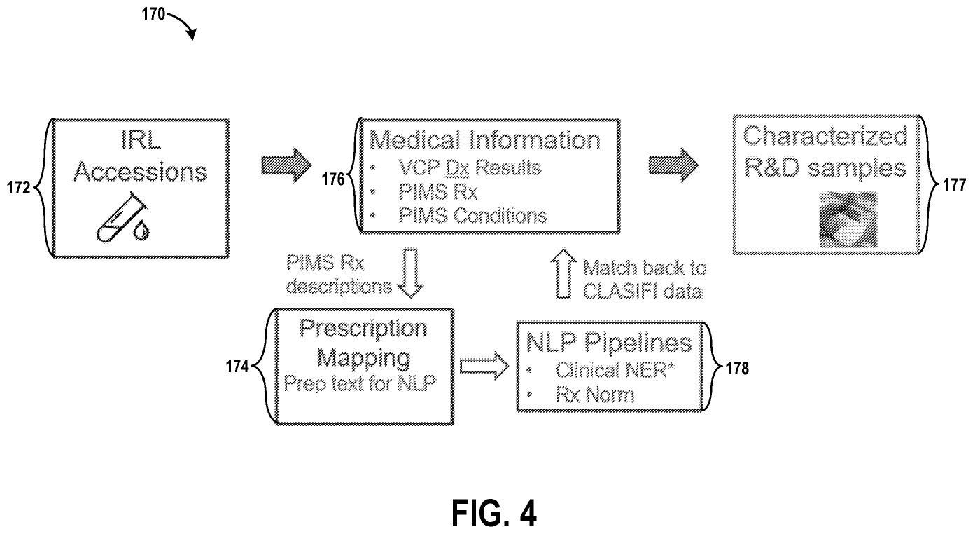

FIGS. 1 and 4 illustrate an example workflow 170 of the functions in FIG. 3, according to an example implementation. In the example workflow 170, test result data 172 for a biological sample at the testing facility 108 for a non-human subject is received at computer system 106. The test result data 172 shown are IDEXX Reference Lab Identification numbers (e.g., IRL Accessions) that are used as the unique sample identifiers in the analysis. The processor 130 of computer system 106 searches within the medical information database 118 to retrieve medical information 176 associated with the subject. The medical information 176 depicted includes diagnostic (Dx) result data (e.g., VetConnect Plus diagnostic result data (VCP Dx Results)), prescription (Rx) data (e.g., PIMS Rx), and condition data (e.g., PIMS Conditions).

In embodiments, the computer system 106 uses the received data to generate prepared data 174 for processing by natural language processing pipelines (e.g., NLP pipelines) 178. In the example shown, prescription drug data is prepared for natural language processing pipelines. To generate prepared data 174 for natural language processing pipelines 178, prescription mapping software performs data processing functions, such as fixing spelling errors, expanding abbreviations, and/or replacing brand name veterinary drugs with ingredient names for mapping to correct controlled unclassified information. The prepared data 174 is fed to the natural language processing pipelines 178 where the prepared data is further processed into a standardized format. In the example shown, the natural language processing pipelines 178 includes clinical named entity recognition (e.g., clinical NER) and Rx Norm, that normalizes the names for clinical drugs and provides a standard clinical drug vocabulary by assigning Rx Norm drug codes. After undergoing the natural language processing pipelines 178, the data is then matched to the retrieved medical information 176 where the data is analyzed to generate characterization for research and development (R&D) samples 177. By processing the data as described above, samples can be categorized into a standardized format and mapped to conditions, allowing for sample characterization to be generated more effectively.

In one example, the processor 130 of computer system 106 replaces brand names of a drug within the medical information 176 with ingredient names of the drug. Based on the ingredient names of the drug, the processor 130 assigns a drug code to the drug. The drug code can be utilized to categorize the biological sample. In some examples, the brand names of the drug are replaced with the ingredient names of the drug by using clinical named entity recognition. In examples, drug code can be an Rx Norm drug code.

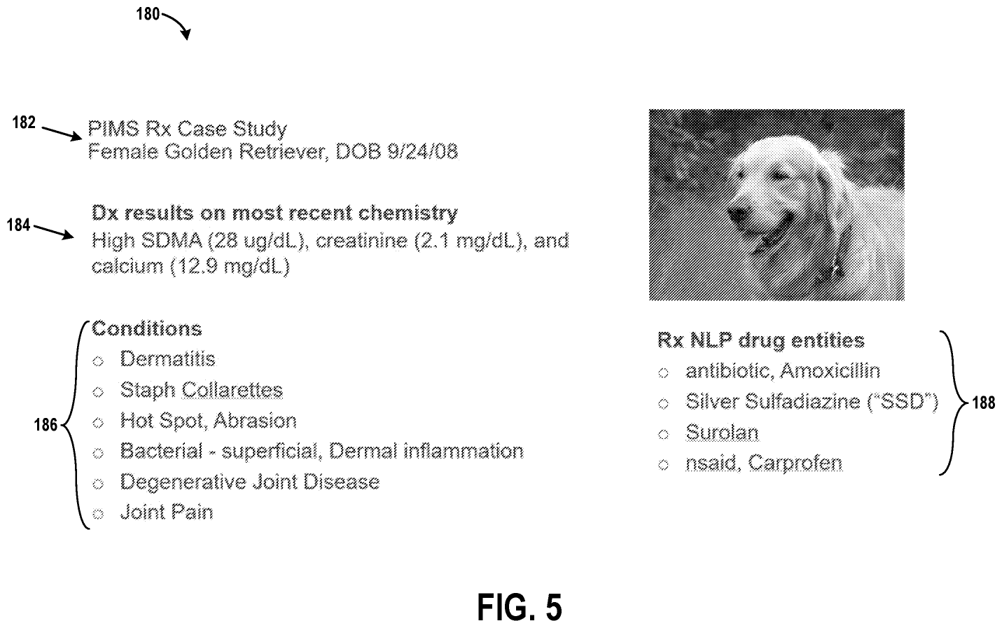

FIG. 5 illustrates sample medical information 180 of the generalized workflow 160 in FIG. 3, according to an example implementation. The sample medical information 180 can be displayed on GUI 114 and may include demographic information 182, diagnostic information 184, condition information 186, and prescription information 188. The demographic information 182 includes species, breed, age, gender, and/or any similar traits of the subject. In the example shown the subject demographic information indicates the non-human subject is a female (e.g., gender), golden retriever (e.g., breed of species Canis familiaris), having a date of birth of Sep. 24, 2008 (e.g., age). While in the view shown in FIG. 5, the demographic information 182 is shown in a structured format, it should be understood that this is merely an example. In embodiments, the demographic information 182 may be embedded in unstructured data, and the first machine-learning algorithm 144 (FIG. 2) can extract the displayed demographic information 182 from the unstructured data.

In embodiments, the computer system 106 receives diagnostic information 184 for the subject in the example of FIG. 5. As shown, diagnostic information 184 indicates the subject has high SDMA levels of 28 μg/dL, creatinine levels of 2.1 mg/dL, and calcium levels of 12.9 mg/dL. The diagnostic information 184 is obtained from one or more tests performed on a biological sample or multiple biological samples from the subject. For example, the diagnostic information 184 of FIG. 5 are test results identified from a blood sample from the subject, however, it should be understood that this is merely an example. In some examples, diagnostic information 184 includes data from a biological tissue sample, an aspirated liquid sample, a liquid blood sample, a dried blood sample, a fecal sample, a urine sample, a saliva sample, a skin swab sample, an ear swab sample, or the like. It should be noted that biological samples are not limited to those mentioned and include any suitable biological sample utilized for testing. While in the view shown in FIG. 5, the diagnostic information 184 is shown in a structured format, it should be understood that this is merely an example. In embodiments, the diagnostic information 184 may be embedded in unstructured data, and the first machine-learning algorithm 144 (FIG. 2) can extract the displayed diagnostic information 184 from the unstructured data.

FIG. 5 includes condition information 186 received by the computer system 106. As shown, the condition information 186 received indicates the subject has dermatitis, staph collarettes, hot spot (abrasion), bacterial—superficial (dermal inflammation), degenerative joint disease, and joint pain. Conditions often have synonyms, and thus reporting conditions often have multiple terms being used to describe the same condition. For example, “bacterial—superficial” in some instances can additionally or alternatively be reported as “dermal inflammation;” and “abrasion” can additionally or alternatively be reported as “hot spot” depending on the reporting terminology. In one example, the processor 130 of computer system 106 searches within a database of the PIMS for the condition information 186. The processor 130 searches within PIMS in a conditions field of a record associated with the subject to extract one or more keywords. Thus, machine-learning natural language processing logic is advantageous in identifying the myriad terminology used in extracting keywords from data containing condition information. While in the view shown in FIG. 5, the condition information 186 is shown in a structured format, it should be understood that this is merely an example. In embodiments, the condition information 186 may be embedded in unstructured data, and the first machine-learning algorithm 144 (FIG. 2) can extract the displayed conditions from the unstructured data.

The example shown in FIG. 5 further includes prescription information 188 received by the computer system 106. The prescription information 188 shown in the example indicates the subject has been or is currently being treated with multiple antibiotics (e.g., Amoxicillin, silver sulfadiazine (SSD), and Surolan), and an NSAID (Carprofen)).

In some examples, information that is useful in diagnosing a non-human subject is found in prescription information sections of the subject's medical information. This information can be found, for example, by the processor 130 of computer system 106 searching within the PIMS for prescription information of medication prescribed to the non-human subject to extract one or more keywords. The prescriptions taken by a non-human subject are indicative of a condition for which the subject is being treated, and/or indicative of a side-effect condition that the subject is experiencing. As an illustrative example, FIG. 5 shows under the prescription information 188 that the subject is prescribed the NSAID Carprofen; and it also shows that under the condition information 186 the subject is suffering from joint pain and degenerative joint disease. As such, the NSAID prescription information can be useful when correlated to the joint condition information of the subject. Chemistry results from a biological sample may be limited in scope and do not reveal a complete medical profile of the subject, such as certain prescription information the subject is taking. Thus, mining of the subject's prescription information is useful in mapping to the subject's biological sample data to create a more complete characterization of the biological sample. While in the view shown in FIG. 5, the prescription information 188 is shown in a structured format, it should be understood that this is merely an example. In embodiments, the prescription information 188 may be embedded in unstructured data, and the first machine-learning algorithm 144 (FIG. 2) can extract the displayed prescription information 188 from the unstructured data.

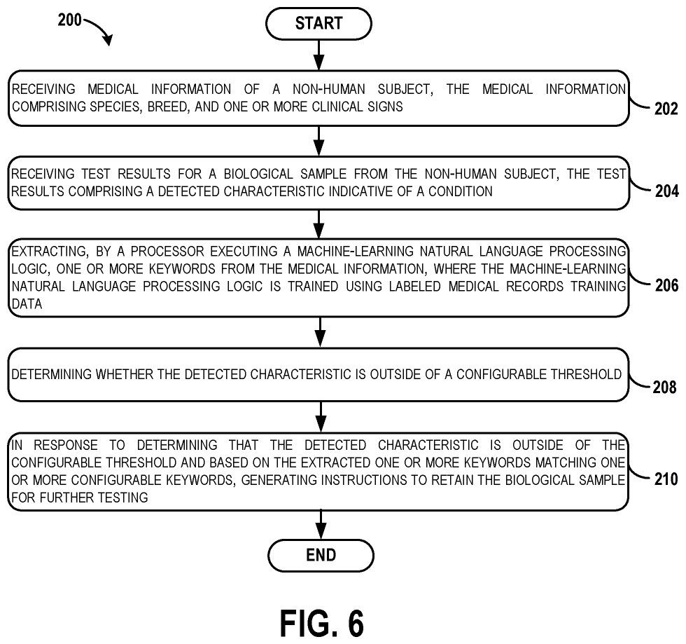

FIG. 6 shows a flowchart of another example of a method 200 for cataloging biological samples from non-human subjects, according to an example implementation. Method 200 shown in FIG. 6 presents an example of a method that could be used with the system 100 shown in FIG. 1, the server 102, shown in FIG. 1, or the computer system 106 shown in FIG. 2, for example. Further, devices or systems may be used or configured to perform logical functions presented in FIG. 6. In some instances, components of the devices and/or systems may be configured to perform the functions such that the components are actually configured and structured (with hardware and/or software) to enable such performance. In some examples, components of the devices and/or systems may be arranged to be adapted to, capable of, or suited for performing the functions, such as when operated in a specific manner. Method 200 may include one or more operations, functions, or actions as illustrated by one or more of blocks 202-210. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long-term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 6, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 202, the method 200 includes receiving medical information of a non-human subject, and the medical information comprising species, breed, and one or more clinical signs. In one example, receiving the medical information in block 202 includes the processor searching within a database of a practice information management system (PIMS) for information in a conditions field of a record associated with the non-human subject to determine the medical information. In another example, receiving the medical information in block 202 includes the processor searching within a database of a practice information management system (PIMS) for prescription information of medication prescribed to the non-human subject to determine the medical information. In one example, receiving the medical information in block 202 comprises receiving clinical information input into a clinical decision support interface on a graphical user interface of a remote computing device for the test results regarding (i) a dose of medication provided to the non-human subject, and (ii) information relating to at least one clinical or historical observation in the non-human subject.

At block 204, the method 200 includes receiving test results for a biological sample from the non-human subject, and the test results comprising a detected characteristic indicative of a condition. In one example, the detected characteristic comprises a detected level of one or more markers including alanine transaminase (ALT). In some examples, the biological sample comprises one or more of a biological tissue sample, an aspirated liquid sample, a liquid blood sample, a dried blood sample, a fecal sample, a urine sample, a saliva sample, and a skin swab sample.

At block 206, the method 200 includes extracting, by a processor executing a machine-learning natural language processing logic, one or more keywords from the medical information, and the machine-learning natural language processing logic is trained using labeled medical records training data. In one example, the keywords comprise a diagnostic code. In some examples, block 206 includes receiving the keywords to be extracted and the configurable threshold from a remote computing device. In another example, extracting the one or more keywords from the medical information at block 206 comprises extracting, by the processor executing a machine-learning natural language processing logic, the one or more keywords from the clinical information.

At block 208, the method 200 includes determining whether the detected characteristic is outside of a configurable threshold.

The method 200 optionally includes determining, by the processor executing a second machine-learning logic, a similarity between the biological sample and other biological samples in the catalog of biological samples exists, and the second machine-learning logic is trained using labeled sample training data. Additionally, for the other biological samples having the similarity, the processor storing within the database data that identifies the other biological samples as being associated with the condition.

At block 210, the method 200 includes in response to determining that the detected characteristic is outside of the configurable threshold and based on the extracted keywords matching one or more configurable keywords, generating instructions to retain the biological sample for further testing. In examples where the test results are associated with a test performed at a first testing location, the method 200 further includes in response to determining that the detected characteristic is outside of the configurable threshold and based on the extracted one or more keywords matching the one or more configurable keywords, generating instructions to send a portion of the biological sample from the first testing location to a second testing location separate from the first testing location.

In some examples, in response to determining that the detected characteristic is outside of the configurable threshold and based on the extracted keywords matching one or more configurable keywords, the method 200 optionally includes generating an electronic consent form for a customer associated with the non-human subject.

The method 200 optionally includes identifying a second biological sample from a same non-human subject from which the biological sample was collected, and generating instructions to retain the second biological sample for further testing. In one example, the second biological sample is of a different type than the biological sample.

In examples where the biological sample has an identifier, the method 200 further includes storing, within a database including a catalog of biological samples, data with the identifier that identifies the biological sample as being associated with the condition and also includes the one or more extracted keywords.

The method 200 optionally includes generating instructions to direct future biological samples from the non-human subject for further analysis.

The method 200 optionally includes replacing, by the processor accessing a drug database, brand names of a drug within the medical information with ingredient names of the drug, based on the ingredient names of the drug, the processor assigning a drug code to the drug, and utilizing the drug code to categorize the biological sample.

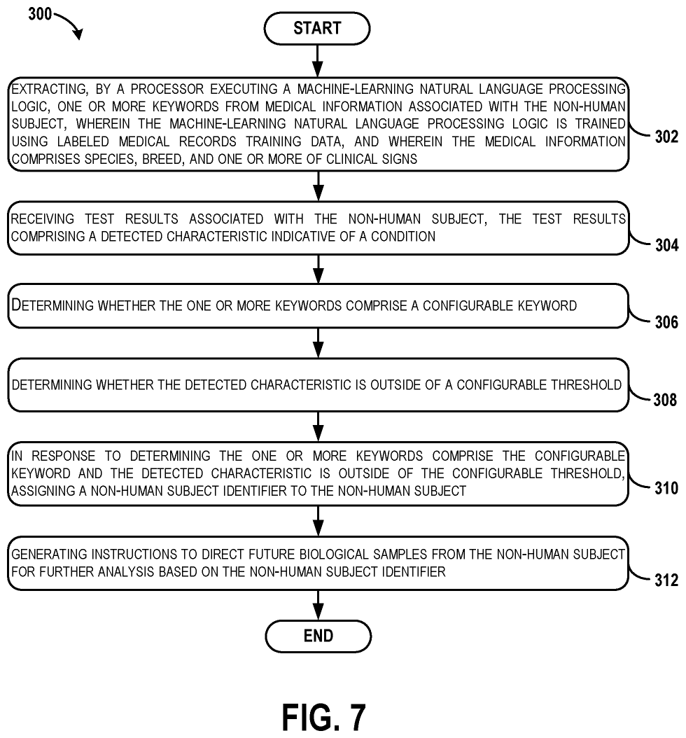

FIG. 7 shows a flowchart of another example of a method 300 for identifying non-human animal non-human subject candidates, according to an example implementation.

At block 302, the method 300 includes extracting, by a processor executing a machine-learning natural language processing logic, one or more keywords from medical information associated with the non-human animal subject, where the machine-learning natural language processing logic is trained using labeled medical records training data, and where the medical information comprises species, breed, and one or more of clinical signs.

At block 304, the method 300 includes receiving test results associated with the non-human animal non-human subject, and the test results comprising a detected level of a component indicative of a condition.

At block 306, the method 300 includes determining whether the one or more keywords comprise a configurable keyword.

At block 308, the method 300 includes determining whether the detected level is outside of a configurable threshold.

At block 310, in response to determining the one or more keywords comprise the configurable keywords and the detected level is outside of the configurable threshold, the method 300 includes assigning a non-human subject identifier to the non-human animal non-human subject.

At block 312, the method 300 includes generating instructions to direct future biological samples from the non-human animal non-human subject for further analysis based on the non-human subject identifier.

The method 300 optionally includes searching, by the processor, within a database of a practice information management system (PIMS) for the medical information in a conditions field of a record associated with the non-human animal non-human subject.

In some examples of method 300 the keywords comprise a diagnostic code that maps to a diagnosis of a biological sample of the non-human animal subject with the condition.

In one example, the detected level of method 300 optionally includes a detected level of one or more markers including alanine transaminase (ALT).

The method 300 optionally includes receiving the medical information as clinical information input into a clinical decision support interface on a graphical user interface of a remote computing device for the test results regarding (i) a dose of medication provided to the non-human subject, and (ii) information relating to at least one clinical or historical observation in the non-human subject; and where extracting the one or more keywords from the medical information comprises extracting, by the processor executing a machine-learning natural language processing logic, the one or more keywords from the clinical information.

With reference to FIG. 2, and throughout the disclosure, some components are described as “modules,” “engines”, “models”, or “generators” and such components include or take a form of a general purpose or special purpose hardware (e.g., general or special purpose processors), firmware, and/or software embodied in a non-transitory computer-readable (storage) medium for execution by one or more processors to perform described functionality.

The description of the different advantageous arrangements has been presented for purposes of illustration and description, and is not intended to be exhaustive or limited to the examples in the form disclosed. Many modifications and variations will be apparent to those of ordinary skill in the art. Further, different advantageous examples may describe different advantages as compared to other advantageous examples. The example or examples selected are chosen and described in order to explain the principles of the examples, the practical application, and to enable others of ordinary skill in the art to understand the disclosure for various examples with various modifications as are suited to the particular use contemplated.

Different examples of the system(s), device(s), and method(s) disclosed herein include a variety of components, features, and functionalities. It should be understood that the various examples of the system(s), device(s), and method(s) disclosed herein may include any of the components, features, and functionalities of any of the other examples of the system(s), device(s), and method(s) disclosed herein in any combination or any sub-combination, and all of such possibilities are intended to be within the scope of the disclosure.

Thus, examples of the present disclosure relate to enumerated clauses (ECs) listed below in any combination or any sub-combination.

EC 1 is a computer-implemented method for cataloging biological samples from non-human subjects, the method comprising: receiving medical information of a non-human subject, the medical information comprising species, breed, and one or more clinical signs; receiving test results for a biological sample from the non-human subject, the test results comprising a detected characteristic indicative of a condition; extracting, by a processor executing a machine-learning natural language processing logic, one or more keywords from the medical information, wherein the machine-learning natural language processing logic is trained using labeled medical records training data; determining whether the detected characteristic is outside of a configurable threshold; and in response to determining that the detected characteristic is outside of the configurable threshold and based on the extracted keywords matching one or more configurable keywords, generating instructions to retain the biological sample for further testing.

EC 2 is the method of EC 1, further comprising searching, by the processor, within a database of a practice information management system (PIMS) for information in a conditions field of a record associated with the non-human subject to determine the medical information.

EC 3 is the method of any of ECs 1-2, further comprising searching, by the processor, within a database of a practice information management system (PIMS) for prescription information of medication prescribed to the non-human subject to determine the medical information.

EC 4 is the method of any of ECs 1-3, wherein: the extracted one or more keywords comprise a diagnostic code.

EC 5 is the method of any of ECs 1-4, further comprising: identifying a second biological sample from a same non-human subject from which the biological sample was collected; and generating instructions to retain the second biological sample for further testing.

EC 6 is the method of any of ECs 1-5, wherein: the second biological sample is of a different type than the biological sample.

EC 7 is the method of any of ECs 1-6, wherein: the biological sample comprises one or more of a biological tissue sample, an aspirated liquid sample, a liquid blood sample, a dried blood sample, a fecal sample, a urine sample, a saliva sample, and a skin swab sample.

EC 8 is the method of any of ECs 1-7, wherein: receiving the medical information comprises receiving clinical information input into a clinical decision support interface on a graphical user interface of a remote computing device for the test results regarding (i) a dose of medication provided to the non-human subject, and (ii) information relating to at least one clinical or historical observation in the non-human subject; and extracting the one or more keywords from the medical information comprises extracting, by the processor executing a machine-learning natural language processing logic, the one or more keywords from the clinical information.

EC 9 is the method of any of ECs 1-8, wherein: the biological sample has an identifier, and the method further comprises: storing, within a database including a catalog of biological samples, data with the identifier that identifies the biological sample as being associated with the condition and also includes the keywords.

EC 10 is the method of any of ECs 1-9, further comprising: determining, by the processor executing a second machine-learning logic, whether a similarity between the biological sample and other biological samples in the catalog of biological samples exists, wherein the second machine-learning logic is trained using labeled sample training data; and for the other biological samples having the similarity, the processor storing within the database data with identifiers of the other biological samples that identifies the other biological samples as being associated with the condition.

EC 11 is the method of any of ECs 1-10, further comprising: generating instructions to direct future biological samples from the non-human subject for further analysis.

EC 12 is the method of any of ECs 1-11, further comprising: receiving the keywords to be extracted and the configurable threshold from a remote computing device.

EC 13 is the method of any of ECs 1-12, wherein: the test results are associated with a test performed at a first testing location, and wherein the computer-implemented method further comprises: in response to determining that the detected characteristic is outside of the configurable threshold and based on the extracted one or more keywords matching the one or more configurable keywords, generating instructions to send a portion of the biological sample from the first testing location to a second testing location separate from the first testing location.

EC 14 is the method of any of ECs 1-13, further comprising: in response to determining that the detected characteristic is outside of the configurable threshold and based on the one or more extracted keywords matching the one or more configurable keywords, generating an electronic consent form for a customer associated with the non-human subject.

EC 15 is the method of any of ECs 1-14, further comprising: replacing, by the processor accessing a drug database, brand names of a drug within the medical information with ingredient names of the drug; based on the ingredient names of the drug, the processor assigning a drug code to the drug; and utilizing the drug code to categorize the biological sample.

EC 16 is a computer-implemented method for identifying non-human subject candidates, the method comprising: extracting, by a processor executing a machine-learning natural language processing logic, one or more keywords from medical information associated with the non-human subject, wherein the machine-learning natural language processing logic is trained using labeled medical records training data, and wherein the medical information comprises species, breed, and one or more of clinical signs; receiving test results associated with the non-human subject, the test results comprising a detected characteristic indicative of a condition; determining whether the one or more keywords comprise a configurable keyword; determining whether the detected characteristic is outside of a configurable threshold; in response to determining the one or more keywords comprise the configurable keywords and the detected characteristic is outside of the configurable threshold, assigning a non-human subject identifier to the non-human subject; and generating instructions to direct future biological samples from the non-human subject for further analysis based on the non-human subject identifier.

EC 17 is the computer-implemented method of EC 16, further comprising: searching, by the processor, within a database of a practice information management system (PIMS) for the medical information in a conditions field of a record associated with the non-human subject.

EC 18 is the computer-implemented method of any of ECs 16-17, wherein: the extracted one or more keywords comprise a diagnostic code associated with a diagnosis of a condition.

EC 19 is the computer-implemented method of any of ECs 16-18, wherein: the detected characteristic comprises a detected level of one or more markers including alanine transaminase (ALT).

EC 20 is the computer-implemented method of any of ECs 16-19, further comprising: receiving the medical information as clinical information input into a clinical decision support interface on a graphical user interface of a remote computing device for the test results regarding (i) a dose of medication provided to the non-human subject, and (ii) information relating to at least one clinical or historical observation in the non-human subject; and wherein extracting the one or more keywords from the medical information comprises extracting, by the processor executing a machine-learning natural language processing logic, the one or more keywords from the clinical information.

EC 21 is a server comprising: one or more processors; and non-transitory computer readable medium having stored therein instructions that when executed by the one or more processors, causes the server to perform functions comprising: receiving medical information of a non-human subject, the medical information comprising species, breed, and one or more clinical signs; receiving test results for a biological sample from the non-human subject, the test results comprising a detected characteristic indicative of a condition; extracting, by the one or more processors executing a machine-learning natural language processing logic, one or more keywords from the medical information, wherein the machine-learning natural language processing logic is trained using labeled medical records training data; and determining whether the detected characteristic is outside of a configurable threshold; and in response to determining that the detected characteristic is outside of the configurable threshold and based on the one or more extracted keywords, generating instructions to retain the biological sample for further testing.

By the term “substantially” and “about” used herein, it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide. The terms “substantially” and “about” represent the inherent degree of uncertainty that may be attributed to any quantitative comparison, value, measurement, or other representation. The terms “substantially” and “about” are also utilized herein to represent the degree by which a quantitative representation may vary from a stated reference without resulting in a change in the basic function of the subject matter at issue.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A computer-implemented method for identifying biological samples from non-human subjects for testing, the method comprising:
receiving medical information of a non-human subject, the medical information comprising species, breed, and one or more clinical signs;
receiving test results for a biological sample from the non-human subject, the test results comprising a detected characteristic indicative of a condition;
extracting, by a processor executing a machine-learning natural language processing logic, one or more keywords from the medical information, wherein the machine-learning natural language processing logic is trained using labeled medical records training data;
determining whether the detected characteristic is outside of a configurable threshold; and
in response to determining that the detected characteristic is outside of the configurable threshold and based on the extracted one or more keywords matching one or more configurable keywords, generating instructions to retain the biological sample for further testing.

2. The computer-implemented method of claim 1, further comprising:
searching, by the processor, within a database of a practice information management system (PIMS) for information in a conditions field of a record associated with the non-human subject to determine the medical information.

3. The computer-implemented method of claim 1, further comprising:
searching, by the processor, within a database of a practice information management system (PIMS) for prescription information of medication prescribed to the non-human subject to determine the medical information.

4. The computer-implemented method of claim 1, wherein the extracted one or more keywords comprise a diagnostic code.

5. The computer-implemented method of claim 4, further comprising:
identifying a second biological sample from a same non-human subject from which the biological sample was collected; and
generating instructions to retain the second biological sample for further testing.

6. The computer-implemented method of claim 5, wherein the second biological sample is of a different type than the biological sample.

7. The computer-implemented method of claim 1, wherein the biological sample comprises one or more of a biological tissue sample, an aspirated liquid sample, a liquid blood sample, a dried blood sample, a fecal sample, a urine sample, a saliva sample, or a skin swab sample.

8. The computer-implemented method of claim 1, wherein:
receiving the medical information comprises receiving clinical information input into a clinical decision support interface on a graphical user interface of a remote computing device for the test results regarding (i) a dose of medication provided to the non-human subject, and (ii) information relating to at least one clinical or historical observation in the non-human subject; and
extracting the one or more keywords from the medical information comprises extracting, by the processor executing a machine-learning natural language processing logic, the one or more keywords from the clinical information.

9. The computer-implemented method of claim 1, wherein the biological sample has an identifier, and the method further comprises:
storing, within a database including a catalog of biological samples, data with the identifier that identifies the biological sample as being associated with the condition and also includes the extracted one or more keywords.

10. The computer-implemented method of claim 9, further comprising:
determining, by the processor executing a second machine-learning logic, whether a similarity between the biological sample and other biological samples in the catalog of biological samples exists, wherein the second machine-learning logic is trained using labeled sample training data; and
for the other biological samples having the similarity, the processor storing within the database data with identifiers of the other biological samples that identifies the other biological samples as being associated with the condition.

11. The computer-implemented method of claim 1, further comprising:
generating instructions to direct future biological samples from the non-human subject for further analysis.

12. The computer-implemented method of claim 1, further comprising:
receiving the one or more keywords to be extracted and the configurable threshold from a remote computing device.

13. The computer-implemented method of claim 1, wherein the test results are associated with a test performed at a first testing location, and wherein the computer-implemented method further comprises:
in response to determining that the detected characteristic is outside of the configurable threshold and based on the extracted one or more keywords matching the one or more configurable keywords, generating instructions to send a portion of the biological sample from the first testing location to a second testing location separate from the first testing location.

14. The computer-implemented method of claim 1, further comprising:
in response to determining that the detected characteristic is outside of the configurable threshold and based on the extracted one or more keywords matching the one or more configurable keywords, generating an electronic consent form for a customer associated with the non-human subject.

15. The computer-implemented method of claim 1, further comprising:
replacing, by the processor accessing a drug database, brand names of a drug within the medical information with ingredient names of the drug;
based on the ingredient names of the drug, the processor assigning a drug code to the drug; and
utilizing the drug code to categorize the biological sample.

16. A computer-implemented method for identifying non-human subject candidates, the method comprising:
extracting, by a processor executing a machine-learning natural language processing logic, one or more keywords from medical information associated with the non-human subject, wherein the machine-learning natural language processing logic is trained using labeled medical records training data, and wherein the medical information comprises species, breed, and one or more of clinical signs;
receiving test results associated with the non-human subject, the test results comprising a detected characteristic indicative of a condition;
determining whether the one or more keywords comprise a configurable keyword;
determining whether the detected characteristic is outside of a configurable threshold;
in response to determining the one or more keywords comprise the configurable keyword and the detected characteristic is outside of the configurable threshold, assigning a non-human subject identifier to the non-human subject; and
generating instructions to direct future biological samples from the non-human subject for further analysis based on the non-human subject identifier.

17. The computer-implemented method of claim 16, further comprising:
searching, by the processor, within a database of a practice information management system (PIMS) for the medical information in a conditions field of a record associated with the non-human subject.

18. The computer-implemented method of claim 17, wherein the extracted one or more keywords comprise a diagnostic code associated with a diagnosis of a condition.

19. The computer-implemented method of claim 17, wherein the detected characteristic comprises a detected level of one or more markers including alanine transaminase (ALT).

20. The computer-implemented method of claim 16, further comprising:
receiving the medical information as clinical information input into a clinical decision support interface on a graphical user interface of a remote computing device for the test results regarding (i) a dose of medication provided to the non-human subject, and (ii) information relating to at least one clinical or historical observation in the non-human subject; and
wherein extracting the one or more keywords from the medical information comprises extracting, by the processor executing a machine-learning natural language processing logic, the one or more keywords from the clinical information.

21. A server comprising:
one or more processors; and
non-transitory computer readable medium having stored therein instructions that when executed by the one or more processors, causes the server to perform functions comprising:
receiving medical information of a non-human subject, the medical information comprising species, breed, and one or more clinical signs;
receiving test results for a biological sample from the non-human subject, the test results comprising a detected characteristic indicative of a condition;
extracting, by the one or more processors executing a machine-learning natural language processing logic, one or more keywords from the medical information, wherein the machine-learning natural language processing logic is trained using labeled medical records training data; and
determining whether the detected characteristic is outside of a configurable threshold; and
in response to determining that the detected characteristic is outside of the configurable threshold and based on the one or more extracted keywords, generating instructions to retain the biological sample for further testing.
